(19) United States
(12) Patent Application Publication
(10) Pub. No.: US 2012/0209124 A1
(43) Pub. Date: Aug. 16, 2012

(54) BREAST HEALTH CARE DEVICE

(76) Inventors: Dar-Bin SHIEH, Tainan City (TW); Pau-Choo CHUNG, Tainan City (TW); Yonhua TZENG, Durham, NC (US)

(21) Appl. No.: 13/354,065

(22) Filed: Jan. 19, 2012

Related U.S. Application Data
(60) Provisional application No. 61/434,863, filed on Jan. 21, 2011.

(30) Foreign Application Priority Data
Oct. 5, 2011 (TW) 100136159
Oct. 5, 2011 (TW) 100136160
Oct. 5, 2011 (TW) 100136161

(51) Int. Cl. A61B 6/00

(52) U.S. Cl. 600/476

(57) ABSTRACT

The present invention relates to a breast health care device, which comprises: a main body, which comprises at least one cover portion; and at least one node module, which is disposed on the cover portion. The node module comprises a light-emitting unit, a photo detector, or the combination thereof, wherein the light-emitting unit provides a light of predetermined wavelength to a breast tissue, and the photo detector detects a first signal generated from the breast tissue. Thus, users can directly achieve detection for the health condition of breasts by wearing the bra of the disclosed breast health care device of the present invention, and the disclosed health care device can transmit the detection results to an external health-care platform such as hospitals or medical centers immediately.

### CROSS REFERENCE TO RELATED APPLICATION

This application claims the benefit of filing date of U.S. Provisional Application Ser. No. 61/434,863, entitled “Breast Health-Care Device” filed Jan. 21, 2011 under 35 USC §119(e)(1).

This application also claims the benefits of the Taiwan Patent Application Serial Number 100136159, 100136160, 100136161, filed on Oct. 5, 2011, the subject matters of which are incorporated herein by reference.

### BACKGROUND OF THE INVENTION

1. Field of the Invention

The present invention relates to a breast health care device and, more particularly, to a breast health care device using near infrared red light. Hence, the users can examine breast tissues periodically to prevent delay in seeking medical care for pathological changes of breasts.

2. Description of Related Art

Breasts are important to a woman's femininity. Many women do not seek medical advice regarding breast issues due to shyness. The importance of effective regular breast examination is usually neglected. Familiar breast diseases comprise: cystic fibrosis, fibroadenoma, intraductal papilloma, breast inflammation, and breast cancer. Other factors such as obesity, fatty food uptake, or radiation in the environment may also increase the risk. The prevention of breast diseases can be improved by effective regular breast self-exam (BSE) to check whether any unusual condition is revealed. In addition, if the excess intake of female hormone drugs is avoided and the contact to the cleanser is reduced, the possibility of suffering from breast diseased can be prevented.

According to the statistics from Department of Health in Taiwan, the incidence and the mortality of breast cancer are increasing year by year. In addition, the ages of the patients are decreasing. These data indicate the need for effective regular screening tool for breast health even at younger age.

There are many methods for checking the health condition of breasts, such as breast self-exam (BSE), palpation, ultrasound examination, breast X-ray mammography and magnetic resonance imaging. However, breast X-ray mammography is discomfort as extreme compression of breast tissue is required. Besides, breast tissues of Asians have higher contents of fibers and usually smaller size, render problematic interpretation of test results and the detection rate. Many women are afraid of palpation and refuse to go to doctors for examination, so breast self-exam (BSE) is performed at home to check the health condition of breasts. However, the lack of essential professional techniques and physical examine background limit the chance of early discovery of unusual breast conditions by patients themselves.

In order to solve the difficulties inherent in the current methods of examination of healthy breasts, the present invention provides a stand-alone BSE assisting device, which is suitable for home care. Hence, ordinary people can check the health condition of breasts regularly. Even though the users do not have professional medical knowledge, they can still perform breast self-exam to check the health condition of their breasts with this device. Hence, women may be willing to perform breast examination regularly, so the purpose of discovery and treatment of breast diseases in early stages may be accomplished. Therefore, the death rate attributed to breast diseases can further be decreased.

### SUMMARY OF THE INVENTION

The object of the present invention is to provide a breast health care device, which is capable of detecting changes in breast tissues with near-infrared light. Since the breast health care device of the present invention is designed into female underwear structure, the users can easily examine the health of their breast tissues by themselves and obtain primary detection results.

To achieve the object, the present invention provides several preferred aspects of breast health care devices. A first preferred aspect of the breast health care device of the present invention comprises: a main body comprising at least one cover portion, wherein the cover portion may have a bra cup structure; and at least one node module disposed on the cover portion, wherein the node module comprises: a light-emitting unit, a photo detector, or a combination thereof. The light-emitting unit provides a light with a predetermined wavelength to breast tissues, and the photo detector detects a first signal generated from the breast tissues. Herein, the first signal can be a photo signal or light intensity.

According to the aforementioned first aspect of the breast health care device of the present invention, the light-emitting unit may be a light emitting diode, a laser light-emitting unit, or a combination thereof. The light-emitting unit can emit light of different wavelengths and different intensities. Preferably, the light-emitting unit emits light with different intensities and with wavelengths located in the near-infrared region. Preferably, the intensities of the light are in the range of 5 mW/cm² to 25 mW/cm². Hence, the breast health care device can detect various breast tissues, from superficial breast tissues, mesoderm breast tissues, to deeper breast tissues, and therefore a better resolution can be obtained. The positions of the node modules disposed on the main body are designed based on the breast structure, in order to detect the optical changes of the breast tissues at each position. In addition, in order to enhance the detection effect, 10-50 node modules may be disposed on each cover portion based on the individuals' breast structure. Preferably, 25-50 node modules are disposed on each cover portion. Furthermore, the node module may further comprise a housing to protect the light-emitting unit, the photo detector such as a phototransistor, or a combination thereof.

The aforementioned first preferred aspect of the breast health care device may further comprise at least one processor, which processes the first signal detected by the photo detector to obtain a second signal. The processor comprises an optical simulation unit such as a Monte Carlo unit to calculate the first signal to obtain the second signal. Herein, the second signal may be a photo signal or an electronic signal, and preferably an electronic signal.

The aforementioned first preferred aspect of the breast health care device may further comprise an information storage module, a signal-transmitting module, or a combination thereof, wherein the information storage module stores the second signal. The signal-transmitting module transmits the second signal. Herein, the signal-transmitting module can transmit the second signal through cable transmission or wireless transmission. The signal-transmitting module may communicate with electronic devices such as smart-phones when these two kinds of transmission are used. Hence, the second signal can be transmitted to the health care platform through the internet to establish a database. Therefore, medical professionals may follow the health conditions of patients with high risk.

In order to make the node modules of the aforementioned first preferred aspect of the breast health care device contact the breast tissues closely, the breast health care device may further comprise a suction unit, an attaching unit, or a combination thereof, wherein the suction unit connects to the node module through a pipe to exhaust gas inside the node module, and the attaching unit is disposed on the node module. The material of the attaching unit may be rubber. Hence, the node module may closely contact the breast tissue. Herein, the suction unit may be an electronic pump or a micro-solenoid valve. Alternatively, the gas inside the node module may be exhausted manually.

Furthermore, the aforementioned first preferred aspect of the breast health care device may further comprise a power supply module, which can provide power to each component of the breast health care device. Therefore, a stand-alone and a portable device can be accomplished.

The aforementioned first preferred aspect of the breast health care device may be customized as female underwear according to the different requirements. Hence, the outmost surface of the main body may be a waterproof surface, so the female underwear can be cleaned several times. In addition, when the node module of the present invention comprise both the light-emitting unit and the photo detector, the material of the female underwear can be any material which can absorb infrared light, UV light or visible light, and preferably is bamboo charcoal fiber. Hence, the material can reduce the interference from other lights near the node module. Therefore, not only can detection be performed on the two sides of the main body of the female underwear, but also the detection effect can further be improved.

A second preferred aspect of the breast health care device of the present invention comprises: a main body comprising at least one cover portion, wherein the cover portion may have a bra cup structure; at least one light-emitting unit disposed on the main body, wherein the light-emitting unit provides a light with predetermined wavelength to breast tissues; and at least one node module disposed on the cover portion, wherein the node module comprises: a photo detector, a light-emitting element, or a combination thereof, wherein the light-emitting element can be an optical fiber for transmitting light with predetermined wavelengths to the breast tissues, the photo detector can be a phototransistor to detect a first signal generated from the breast tissues. Herein, the first signal can be a photo signal or light intensity.

According to the aforementioned second aspect of the breast health care device of the present invention, the light-emitting unit may be a light emitting diode, a laser light-emitting unit, or a combination thereof. The light-emitting unit can emit light with at least one predetermined wavelength to detect breast tissues. Herein, the light-emitting unit can be designed as a rotative light-emitting unit, or a linear reciprocating light-emitting unit. When the light-emitting unit is a rotative light-emitting unit, original: light-emitting heads may surround the rotative light-emitting unit. Hence, the rotative light-emitting unit may correspond to different light transmitting units at different time points, so light can be emitted to each different node module sequentially. When the light-emitting unit is a linear reciprocating light-emitting unit, the light with different wavelengths may be transmitted to different light-emitting element according to the predetermined time sequence, to transmit the light to different node modules. With regard to the wavelength and the intensity of the light emitting from the light-emitting unit, the wavelength thereof is preferably located within the near-infrared region, and the intensity thereof is preferably in the range of 5 mW/cm² to 25 mW/cm². In addition, when the light with different intensities and wavelengths illuminates to the breast tissues corresponding to the node module, the effect on the detection of breast tissues can further be improved.

According to the aforementioned second aspect of the breast health care device of the present invention, the positions of the node modules disposed on the main body are designed based on the breast structure, in order to detect the optical changes of the breast tissues on each position. In addition, in order to enhance the detection effect, 10-50 node modules may be disposed on each cover portion based on the individuals' breast structure. Preferably, 25-50 node modules are disposed on each cover portion. Furthermore, the node module may further comprise a housing to protect the photo detector, the light-emitting element, or a combination thereof, wherein the light-emitting unit may be an optical fiber, and the photo detector may be a phototransistor.

The aforementioned second preferred aspect of the breast health care device may further comprise at least one processor, which processes the first signal detected by the photo detector to obtain a second signal. The processor comprises an optical simulation method such as a Monte Carlo unit, to calculate the first signal to obtain the second signal. Herein, the second signal may be a photo signal or an electronic signal, and preferably an electronic signal.

The aforementioned second preferred aspect of the breast health care device may further comprise an information storage module, a signal-transmitting module, or a combination thereof to store the second signal and transmit the second signal to a health care platform for follow up. In addition, the breast health care device may further comprise a suction unit, an attaching unit, or a combination thereof to enhance the effect on the detection of the breast health care device. The configurations of the suction unit and the attaching unit are the same as the aforementioned device. Furthermore, the second preferred aspect of the breast health care device may be customized as female underwear according to different requirements. The material of the surface of the cover portion and the cover portion itself can be waterproof material and a material that can absorb infrared light, UV light or visible light, in order to clean the breast health care device and improve the detection effect thereof. In addition, the breast health care device may further comprise a power supply module, which can provide power to each component of the breast health care device. Therefore, a portable device can be accomplished.

In order to simplify the structure of the breast health care device of the present invention, a third preferred aspect of the breast health care device is also provided, which integrates the light-emitting unit and the photo detector. The third preferred aspect of the breast health care device comprises: a main body comprising at least one cover portion, wherein the cover portion may have a bra cup structure; a light-emitting/detecting unit disposed on the main body, wherein the light-emitting/detecting unit provides light with a predetermined wavelength to breast tissues and detects a first signal generated from the breast tissues, and the first signal can be a photo signal or light intensity; and at least one node module disposed on the cover portion, wherein the node module comprises: a light transmitting unit, a photo signal transmitting unit, or a combination thereof, wherein the light transmitting unit transmits light with a predetermined wavelength to the breast tissues, and the photo signal transmitting unit transmits the first signal to the light-emitting/detecting unit.

According to the aforementioned third aspect of the breast health care device of the present invention, the light-emitting/detecting unit may be a rotative light-emitting/detecting unit or a linear reciprocating light-emitting/detecting unit. When the light-emitting/detecting unit is a rotative light-emitting/detecting unit, light-emitting heads may surround the rotative light-emitting/detecting unit. Hence, when the rotative light-emitting/detecting unit rotates, light can transmit to different node modules through the light-transmitting unit at different time points, and then the first signal generated from the breast tissues may transmit back to a photo detector of the light-emitting/detecting unit through the photo signal-transmitting unit of the node module. After the aforementioned process, the detection is completed. When the light-emitting/detecting unit is a linear reciprocating light-emitting/detecting unit that is different from the rotative light-emitting/detecting unit, the light-emitting units are linearly arranged. Hence, when the light-emitting units move linearly, light with different wavelengths may be transmitted to different light-transmitting units according to the time sequence setting, transmitting light to different node modules. Then, the first signal generated from the breast tissues may transmit back to the linear reciprocating light-emitting/detecting unit through the photo signal-transmitting unit of the node module to complete the detection. The light-emitting unit of the light-emitting/detecting unit is a light emitting diode, a laser light-emitting unit, or a combination thereof, which can emit light with at least one predetermined wavelength, and the wavelength thereof is preferably located within the near-infrared region. The light emitting diode may further emit light with different intensities, which are preferably in the range of 5 mW/cm² to 25 mW/cm². When the light with different intensities and wavelengths illuminates to the breast tissues, the detection may be performed on the breast tissues at different depths on the breast tissues to improve the detection effect of the breast health care device.

According to the aforementioned third aspect of the breast health care device of the present invention, at least one of the light-transmitting units and the photo signal-transmitting units is an optical fiber. When the detection is performed, the light-transmitting unit is used to transmit light emitting from the light-emitting/detecting unit to the node module, and the photo signal-transmitting unit is used to transmit the first signal generated from the breast tissues back to the light-emitting/detecting unit.

The aforementioned third preferred aspect of the breast health care device may further comprise at least one processor, which processes the first signal detected by the light-emitting/detecting unit to obtain a second signal. The processor comprises an optical simulation unit such as a Monte Carlo unit, to calculate the first signal to obtain the second signal. Herein, the second signal may be a photo signal or an electronic signal, and preferably an electronic signal.

In addition, the aforementioned third preferred aspect of the breast health care device may further comprise an information storage module, a signal-transmitting module, or a combination thereof, which can store the second signal and transmit the second signal to a health care platform to establish a database for follow up. In addition, the aforementioned third preferred aspect of the breast health care device may further comprise a suction unit, an attaching unit, or a combination thereof, which can enhance the attachment between the node module and the breast tissues to improve the detection effect of the breast health care device. Furthermore, the third preferred aspect of the breast health care device may be customized as female underwear according to different requirements. The material of the surface of the cover portion and the cover portion itself can be a waterproof material for easy cleaning and a material that can absorb infrared light, UV light or visible light. Finally, the breast health care device may further comprise a power supply module, which can provide power to each component of the breast health care device. The aforementioned components may be the same as those described above.

In conclusion, the breast health care device of the present invention can abolish the pain and the inconvenience when women perform breast examinations. The breast health care device of the present invention is designed as female underwear. When users wear the breast health care device with a bra structure, the primary health condition of the breast can be determined through the change of optical characteristics. In addition, the detection results can be transmitted to computers or health care platforms through the processor and the signal-transmitting module. Through the use of this device, the convenience for the detection of the breast health condition can be improved. Furthermore, light with wavelengths in the near infrared red region is used to detect the breast tissues in the breast health care device of the present invention, so the effect of enhancing blood circulation can also be accomplished. Moreover, when the light-emitting unit of the breast health care device of the present invention is a rotative light-emitting unit, light with different colors may also be combined therein to obtain a varied visual effect.

### BRIEF DESCRIPTION OF THE DRAWINGS

FIG. 1 is a perspective view of a breast health care device of Embodiment 1 of the present invention;

FIG. 2 is a cross-sectional view of a breast health care device of Embodiment 1 of the present invention;

FIG. 3 is a perspective view showing the structure of a node module of Embodiment 1 of the present invention;

FIG. 4 is a perspective view showing the structure of a node module of Embodiment 1 of the present invention;

FIG. 5 is a perspective view showing the structure of a node module of Embodiment 2 of the present invention;

FIG. 6 is a perspective view of a breast health care device of Embodiment 3 of the present invention;

FIG. 7 is a cross-sectional view of a breast health care device of Embodiment 3 of the present invention;

FIG. 8 is a perspective view showing the structure of a node module of Embodiment 3 of the present invention;

FIG. 9 is a perspective view showing the structure of a rotative light-emitting unit of Embodiment 3 of the present invention;

FIG. 10 is a perspective view of a breast health care device of Embodiment 4 of the present invention;

FIG. 11 is a perspective view showing the structure of a node module of Embodiment 4 of the present invention;

FIG. 12 is a perspective view showing the structure of a linear reciprocating light-emitting unit of Embodiment 4 of the present invention;

FIG. 13 is a perspective view of a breast health care device of Embodiment 4 of the present invention;

FIG. 14 is a perspective view showing the structure of a node module of Embodiment 5 of the present invention;

FIG. 15 is a perspective view showing the structure of a rotative light-emitting/detecting unit of Embodiment 5 of the present invention;

FIG. 16 is a perspective view showing the structure of a rotative light-emitting/detecting unit of Embodiment 6 of the present invention; and

FIG. 17 is a perspective view showing the structure of a linear reciprocating light-emitting/detecting unit of Embodiment 7 of the present invention.

### DETAILED DESCRIPTION OF THE PREFERRED EMBODIMENT

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

### Embodiment 1

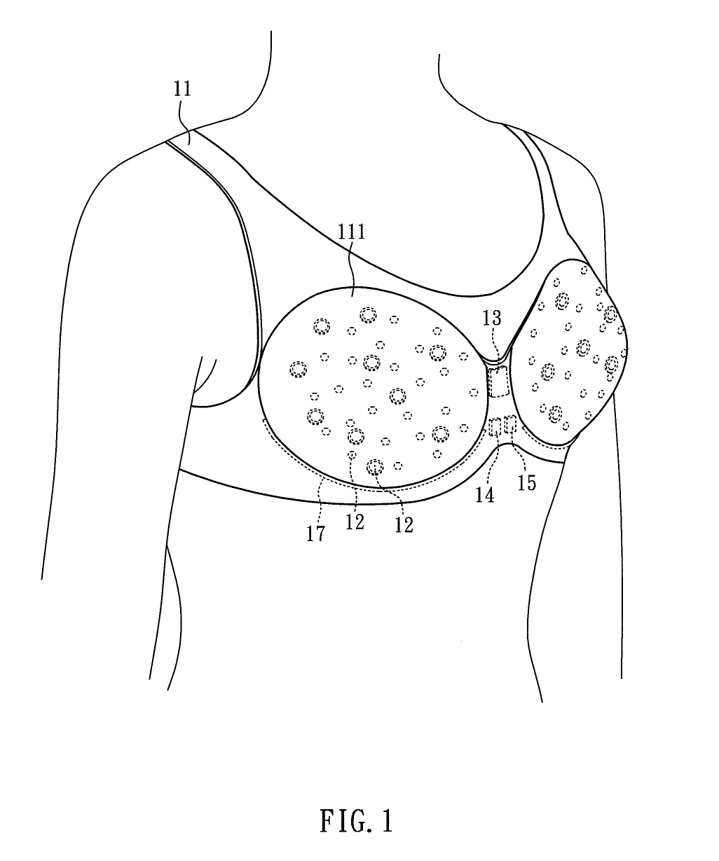

As shown in FIGS. 1-4, the breast health care device of the present embodiment comprises: a main body 11 comprising two cover portions 111, wherein the cover portions 111 have bra cup structures. Eighteen node modules 12 are respectively disposed on the right and the left cover portion 111.

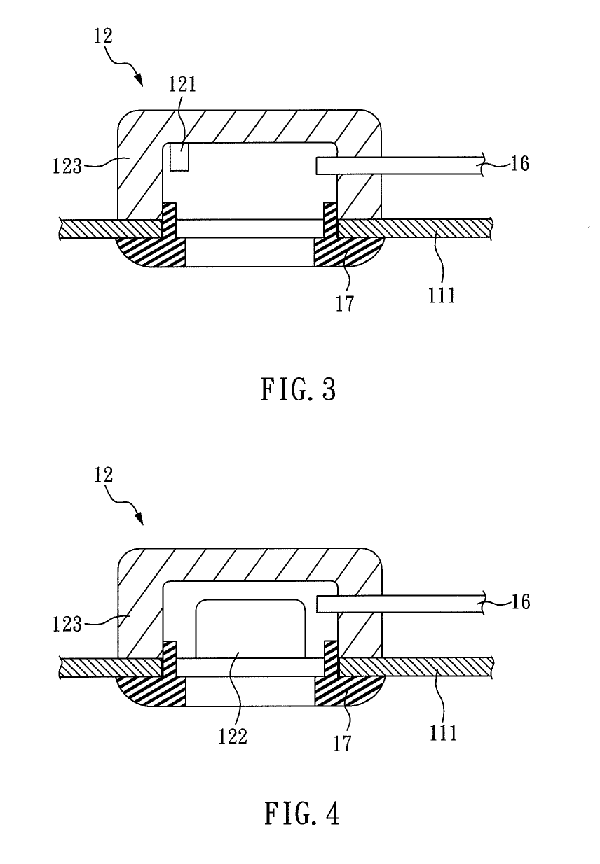

In the present embodiment, the structures of the node modules 12 are classified into two types. One type of node module 12 is a node module only provided with a light-emitting unit 121 (as shown in FIG. 3), and the other type is a node module only provided with a photo detector 122 (as shown in FIG. 4). The light-emitting unit 121 provides a light with predetermined wavelength to breast tissues, and the photo detector 122 detects a first signal generated from the breast tissues. Herein, the first signal is a light intensity.

As shown in FIG. 1, the breast health care device of the present embodiment comprises a processor 13, an information storage module 14 and a signal-transmitting module 15. The processor 13 processes the first signal detected by the photo detector of the node module 12, and then the first signal is calculated with a Monte Carlo unit to obtain a second signal. Herein, the second signal is an electronic signal. In addition, the information storage module 14 stores the aforementioned second signal, and the signal-transmitting module 15 only transmits the second signal. In the present embodiment, the signal-transmitting module 15 transmits the second signal through wireless transmission. Hence, the signal-transmitting module 15 transmits the processed signal to an electronic device through an antenna 18. Then, the signal is sequentially transmitted to a health care platform through the internet to establish a database, so medical professionals may follow the health conditions of patients with high risk.

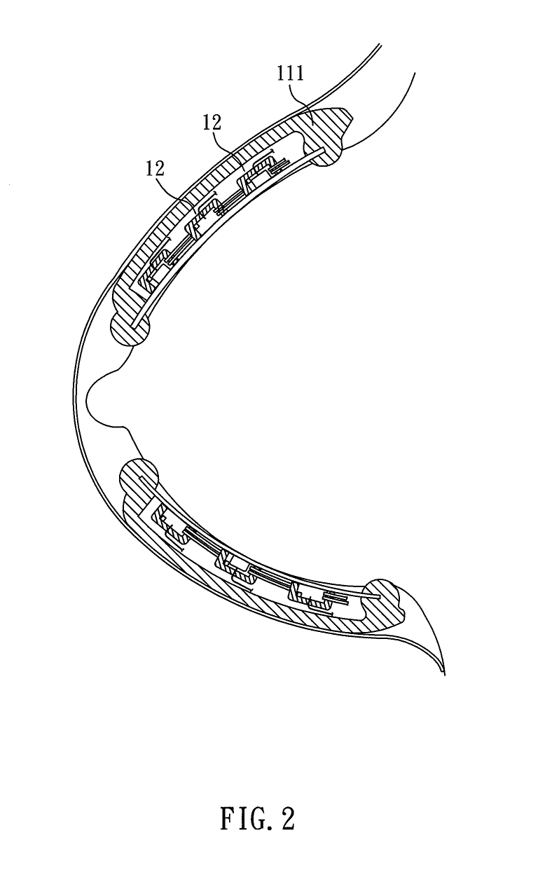

FIG. 2 is a cross-sectional view of a breast health care device of the present embodiment. As shown in FIG. 2, the node modules 12 with the light-emitting unit, and the node modules 12 with the photo detector are uniformly dispersed on the cover portion 111. The first signal generated from the breast tissues may be accepted by the surrounding photo detector of the node module 12, so the detection of the breast health care device can be accomplished.

FIG. 3 shows the node module 12 provided with a light-emitting unit 121, wherein the light-emitting unit 121 is a light emitting diode (LED). The wavelength of the LED is located in the near-infrared region, and the intensity thereof is in the range of 5 mW/cm² to 25 mW/cm². In addition, the wavelength and the intensity thereof can be modified based on the detection requirement. The node module 12 is provided with a housing 123, and the light-emitting unit 121 is disposed in the housing 123. In addition, the node module 12 is provided with a suction unit 16 and an attaching unit 17. The suction unit 12 removes gas inside the housing 123 through a pipe, and the node module 12 closely contacts the breast tissue through the attaching unit 16. Herein, the suction unit 16 is an electronic pump, and the material of the attaching unit 17 is rubber.

FIG. 4 shows a node module 12 provided with a photo detector 122, wherein the photo detector 122 is a phototransistor. The node module 12 is also provided with a housing 123, and the photo detector 122 is disposed in the housing 123. In addition, the node module 12 is also provided with a suction unit 16 and an attaching unit 17. The suction unit 12 removes gas inside the housing 123 through a pipe, and the node module 12 closely contacts the breast tissue through the attaching unit 16. Herein, the suction unit 16 is a micro-solenoid valve, and the material of the attaching unit 17 is rubber.

Furthermore, the breast health care device of the present embodiment is provided with a power supply (not shown in the figure) to provide power to each component of the breast health care device. Therefore, a stand-alone and portable device can be accomplished.

In order to use conveniently, the breast health care device of the present invention is designed as female underwear. The outmost surface of the main body may be a waterproof surface, so the female underwear can be cleaned several times without destroying the inner components of the breast health care device. In addition, the material of the cover portion of the breast health care device of the present embodiment is bamboo charcoal fiber, which has light absorption properties. Hence, the material can reduce the interference from other lights near the node module. The breast health care device of the present embodiment can detect two sides of the breast tissues at the same time, so the time for detection can be decreased further.

### Embodiment 2

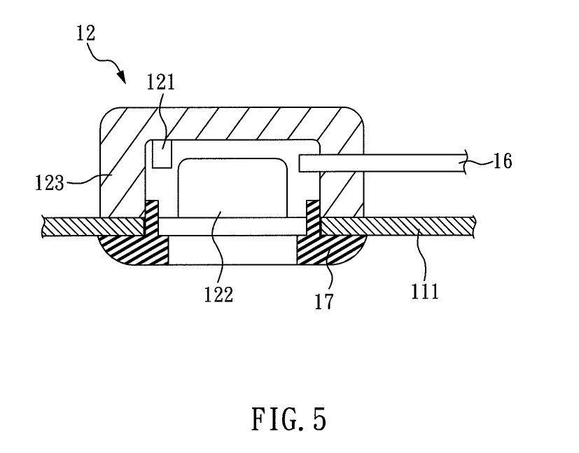

The structure of the breast health care device of the present embodiment is almost the same as that disclosed in Embodiment 1, except that the structure of the node module 12 is different. FIG. 5 is a perspective view showing the structure of a node module of the present embodiment. The node module of the present embodiment comprises a light-emitting unit 121 (LED), a photo detector 122, and a housing 123. In addition, the node module also comprises a suction unit 16 and an attaching unit 17.

In the present embodiment, the light-emitting unit 121 and the photo detector 122 are integrated into a node module 122. Hence, when the light-emitting unit 122 emits near infrared light with different wavelengths and different intensities (5 mW/cm² to 25 mW/cm²), the photo detector 122 can detect the first signal generated from the breast tissues, which correspond to this node module 121.

### Embodiment 3

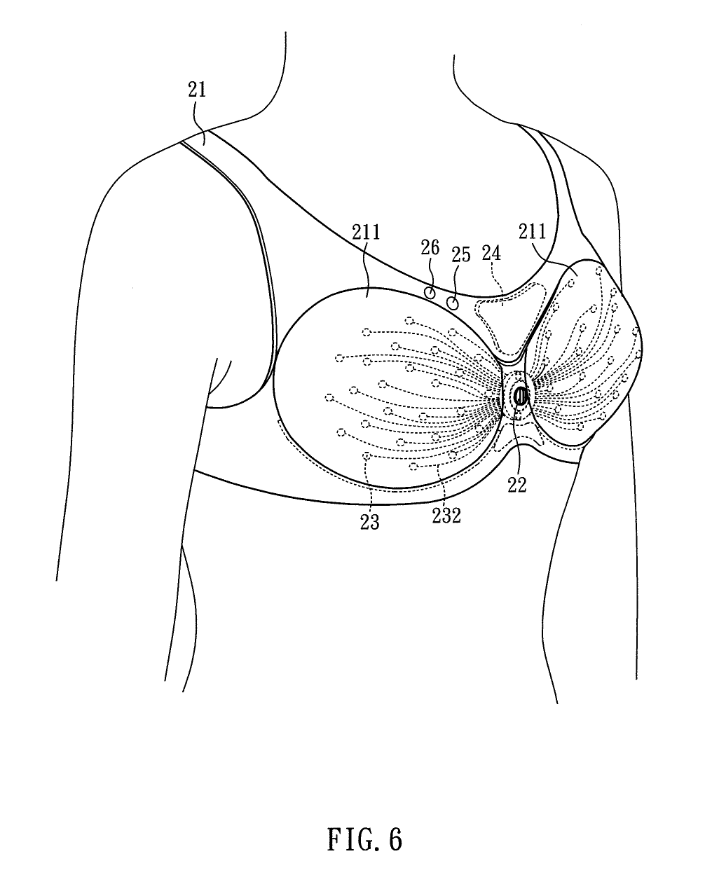

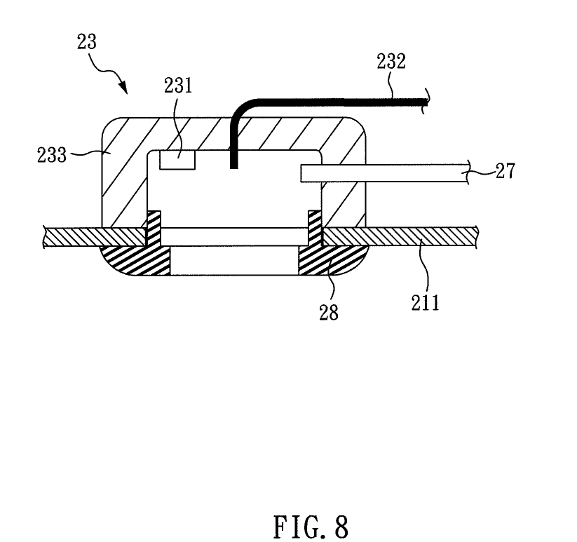

As shown in FIGS. 6-8, the breast health care device of the present embodiment comprises: a main body 21 comprising at least one cover portion 211, wherein the cover portions 211 have bra cup structures; a light-emitting unit 22 disposed on the center of the main body 21, wherein the light-emitting unit 22 provides light with a predetermined wavelength to breast tissues; and forty node modules 23 disposed on the cover portions 211 on two sides, wherein the node module 23 comprises a photo detector 231 and a light-transmitting unit 232, as shown in FIG. 8. The light-transmitting unit 232 transmits the light emitting from the light-emitting unit 22, and the photo detector 231 detects a first signal generated from the breast tissues. Herein, the first signal is light intensity.

As shown in FIG. 6, the breast health care device of the present embodiment comprises a processor 24, an information storage module 25 and a signal-transmitting module 26. The processor 24 processes the first signal detected by the photo detector 231 of the node module 12, and then the first signal is calculated with a Monte Carlo unit to obtain a second signal. Herein, the second signal is an electronic signal. In addition, the information storage module 25 stores the second signal, and the signal-transmitting module 26 transmits the second signal. In the present embodiment, the signal-transmitting module 15 transmits the second signal through cable transmission. Hence, the signal-transmitting module 15 transmits the processed signal to a smartphone through USB. Then, the signal is sequentially transmitted to a health care platform through the internet to establish a database.

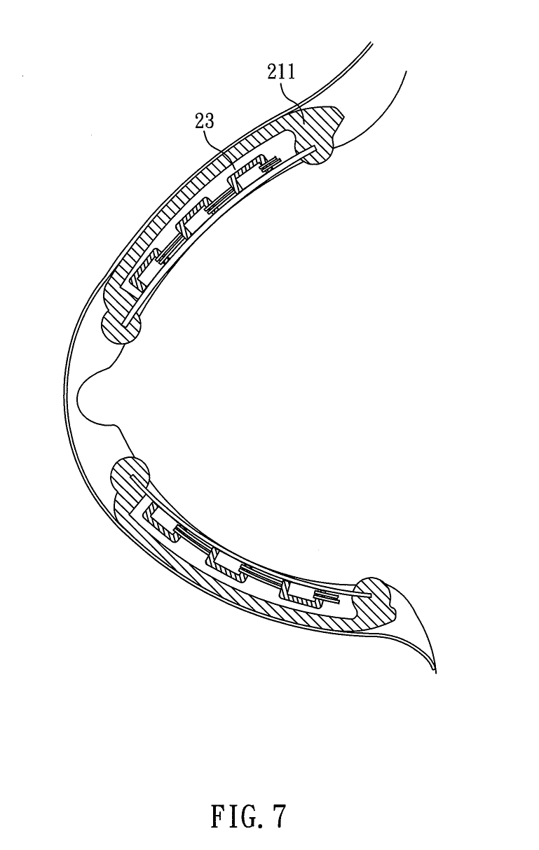

FIG. 7 is a cross-sectional view of a breast health care device of the present embodiment. As shown in FIG. 7, the positions of the node modules 23 are designed according to the individuals breast structure, so a personal detection on the breast tissue can be accomplished.

FIG. 8 is a perspective view showing the structure of a node module 23 of the present embodiment. The node module 23 comprises the photo detector 231 and the light-transmitting unit 232 disposed in the housing 233. The photo detector 231 is a phototransistor, and the light-transmitting unit 232 is an optical fiber. In addition, the node module 23 is also provided with a suction unit 27 and an attaching unit 28. The suction unit 27 removes gas inside the housing 233 through a pipe, and the node module 23 closely contacts the breast tissue through the attaching unit 28. Herein, the suction unit 27 is an electronic pump, and the material of the attaching unit 28 is rubber.

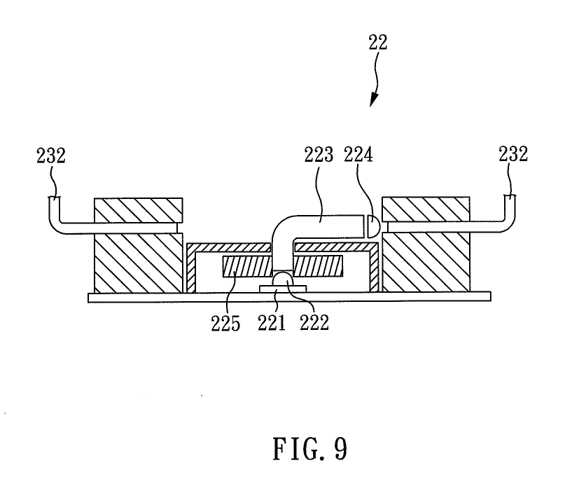

FIG. 9 is a perspective view showing the structure of the light-emitting unit of the present embodiment 3. The light-emitting unit of the present embodiment is a rotative light-emitting unit, which comprises a light emitting diode (LED) 221, a first lens 222, a light-transmitting unit 223 being an optical fiber in the present embodiment, a second lens 225, and a rotating unit 225. The light emitting diode 221 can emit lights with different wavelengths and intensities. When the light emitting diode 221 emits light, the light is focused through the first lens 222 and enters into the light-transmitting unit 223. Next, the light-transmitting unit 223 transmits the focused light to the second lens 224 to perform another focus process, and then the focused light enters into the optical fiber 232. The rotating unit 225 of the rotative light-emitting unit can change the positions of the light-transmitting unit 223 and the second lens 224, to make the light transmitting to different node modules 23. Herein, the light emitting from the light emitting diode 221 can pass through the first lens 222, the light-transmitting unit 223 and the second 224 rotating unit 225 and sequentially arrive to each different node module 23 based on the time sequence setting for controlling the rotating unit 225 of the rotative light-emitting unit.

In addition, the breast health care device of the present embodiment is provided with a power supply (not shown in the figure), which is disposed on the buckle of the breast health care device. Herein, the battery used in the power supply can be a lithium secondary battery, in order to reduce the weight of the device of the present embodiment. In addition, the breast health care device of the present invention is designed as female underwear, and the material thereof is a waterproof material and bamboo charcoal fibers. Hence, not only can the device of the present embodiment be cleaned several times, the detection effect thereof can be further improved.

### Embodiment 4

The structure of the breast health care device of the present embodiment is almost the same as that disclosed in Embodiment 1, except that the structures of the node module and the light-emitting unit are different from those disclosed in Embodiment 3.

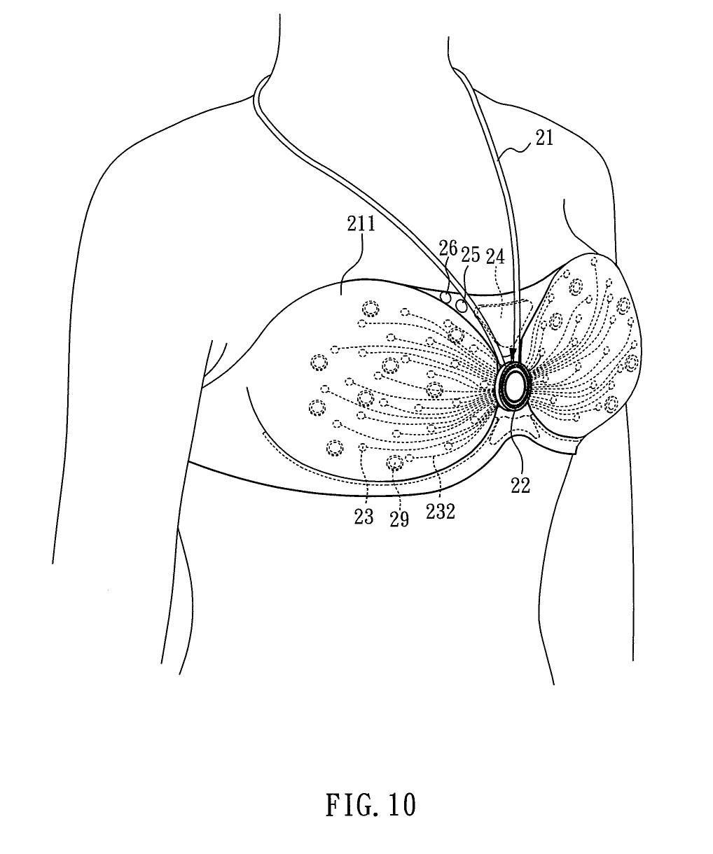

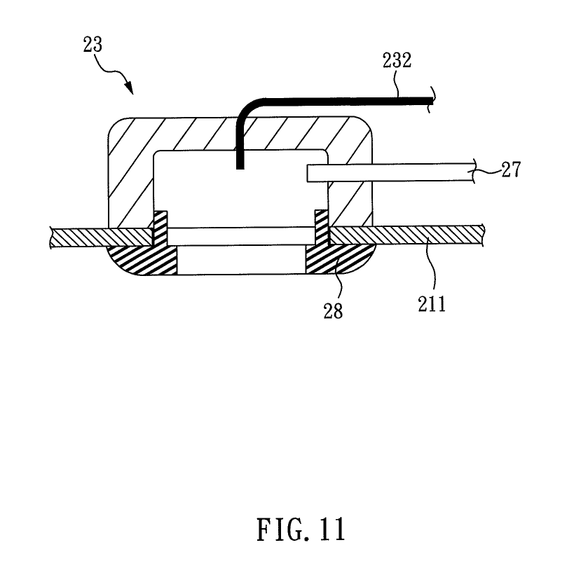

As shown in FIGS. 10-11, the structure of the node module of the present embodiment is almost the same as that of Embodiment 3, except that the device of the present embodiment comprises two types of node modules, wherein one type of node module 23 is a node module only provided with a light-emitting unit 232 (fiber) and without a photo detector (as shown in FIG. 11), and the other type is a node module 29 only provided with a photo detector (as shown in FIG. 10). The node module 29 can detect a first signal generated from the breast tissues surrounding the node module 23, to accomplish the purpose of the detection. Herein, the first signal is a photo signal.

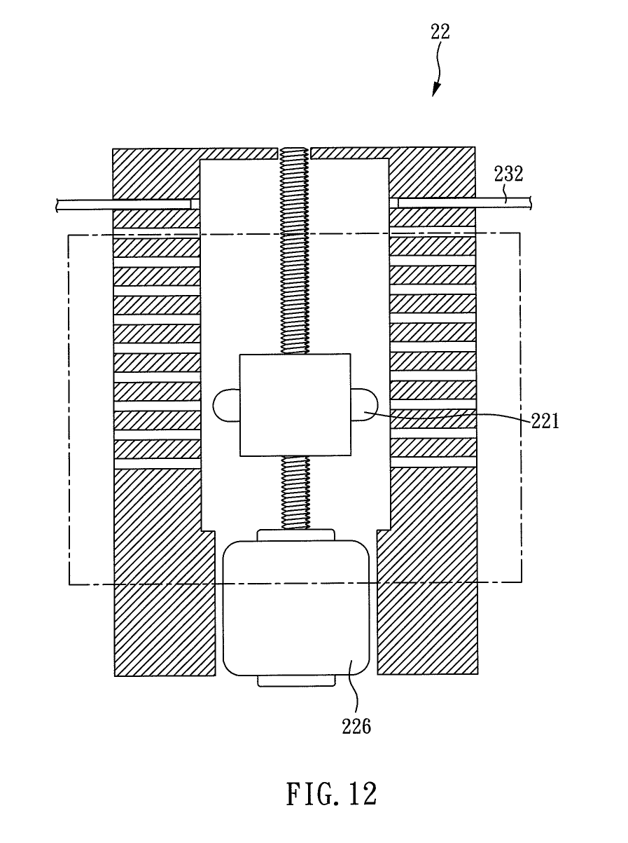

In addition, as shown in FIG. 12, the light-emitting unit of the present embodiment is a linear reciprocating light-emitting unit, which comprises a motor 225, two light emitting units (LED) 221, and light-transmitting element 232 such as optical fibers. Herein, the motor 225 is used to drive the light-emitting units 221 to make the light-emitting unit move linearly and reciprocatingly, so the light emitting from the light-emitting units 221 can arrive to each different node module through the light-transmitting units 232. In the linear reciprocating light-emitting unit of the present embodiment, the light with different wavelengths and intensities generated from the light-emitting units 221 can transmit to each different node module based on the setting of the time sequence.

### Embodiment 5

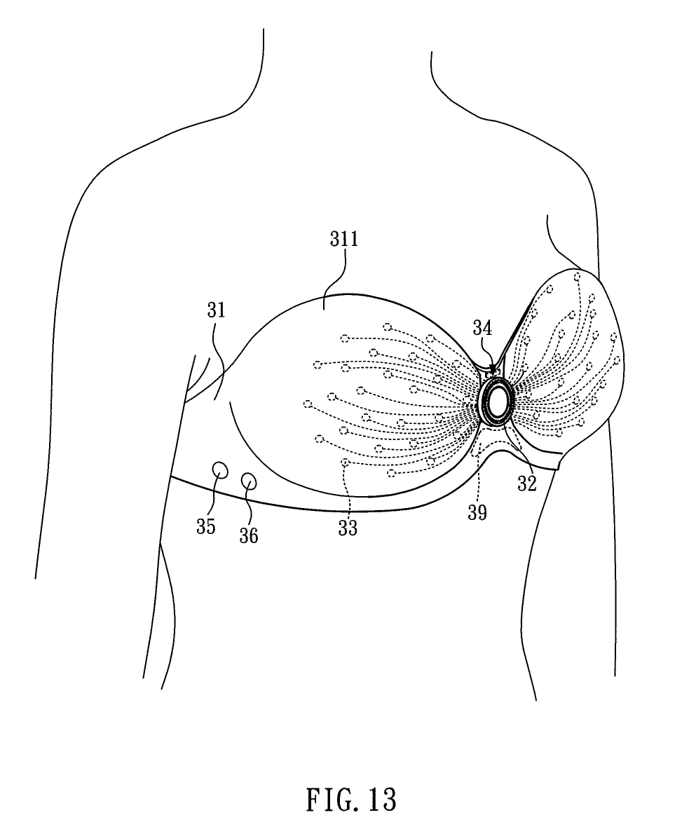

As shown in FIGS. 13-15, the breast health care device of the present embodiment comprises: a main body 31 comprising at least one cover portion 311, wherein the cover portions 311 have bra cup structures; a light-emitting/detecting unit 32 disposed on the center of the main body 31, wherein the light-emitting/detecting unit 32 provides light with a predetermined wavelength to breast tissues and detects a first signal (i.e. light intensity) generated from the breast tissues; and at least one node module 33 disposed on the cover portion 331, wherein the node module 33 comprises a light-transmitting unit 331 and a photo signal-transmitting unit 332. The light-transmitting unit 331 transmits light with a predetermined wavelength to the breast tissues, and the photo signal-transmitting unit 332 transmits the first signal to the light-emitting/detecting unit 32 to accomplish the purpose of breast detection.

As shown in FIG. 13, the breast health care device of the present embodiment comprises a processor 34, an information storage module 35 and a signal-transmitting module 35. The processor 34 processes the first signal detected by the light-emitting/detecting unit 32, and then the first signal is calculated with a Monte Carlo unit to obtain a second signal such as an electronic signal. In addition, the information storage module 35 stores the aforementioned second signal, and the signal-transmitting module 36 transmits the second signal. In the present embodiment, the second signal is transmitted to a smart-phone through wireless transmission, and the signal is sequentially transmitted to a health care platform through the internet to establish a database. In addition, the breast health care device of the present embodiment is provided with a power supply 39 to provide power to each component of the breast health care device. Therefore, a portable device can be accomplished.

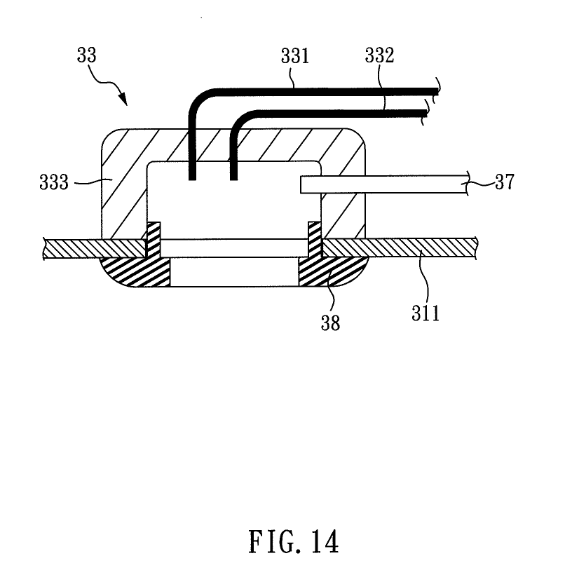

FIG. 14 is a perspective view showing the structure of a node module of Embodiment 5 of the present invention. As shown in FIG. 14, the node module 33 comprises: a light-transmitting unit 331, a photo signal-transmitting unit 332, and a housing 333. Herein, the light-transmitting unit 331 and the photo signal-transmitting unit 332 are optical fibers. The light-transmitting unit 331 transmits the light generated from the light-emitting/detecting unit 32 to the node module 33, and the photo signal-transmitting unit 332 transmits the light generated from the breast tissues back to the light-emitting/detecting unit 32, to perform the detection. In addition, the node module 33 is also provided with a suction unit 37 and an attaching unit 38. The suction unit 37 removes gas inside the housing 333 through a pipe, and the node module 33 closely contacts the breast tissue through the attaching unit 38. In the present embodiment, the suction unit 37 is an electronic pump, and the material of the attaching unit 38 is silica gel.

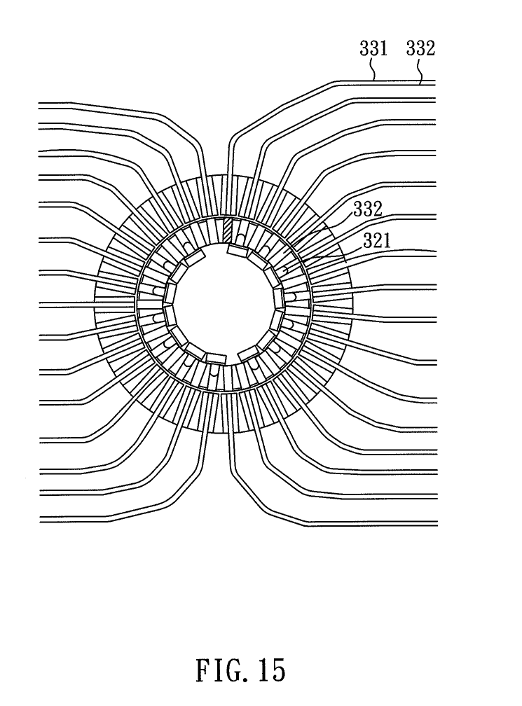

FIG. 15 is a perspective view showing the structure of a rotative light-emitting/detecting unit of Embodiment 5 of the present invention. As shown in FIG. 15, the light-emitting/detecting unit of the present embodiment is a rotative light-emitting/detecting unit 32, which comprises a light-emitting unit 321 and a photo detector 322. Herein, the light-emitting unit 321 and the photo detector 322 connect to the light-emitting/detecting unit 32 to transmit or receive photo signals.

The rotative light-emitting/detecting unit 32 of the present embodiment comprises fourteen light-emitting units 321 such as LEDs and plural photo detectors 322 such as phototransistors. Herein, seven light-emitting units 321 are arranged linearly. On the left side and the right side are disposed a set of light-emitting units 321 with different wavelengths and intensities, and each light-emitting unit 321 corresponds to a different light-transmitting unit 331 such as an optical fiber. Then, the light is transmitted to each node module 33 through the light-transmitting unit 331. Herein, each photo detector 322 is respectively disposed between the light-emitting units 321, and the first signal generated from the breast tissues corresponding to the node module 33 is transmitted through the photo signal-transmitting unit 332 to finish the detection. In order to detect accurately, each light-emitting unit 321 emits light with different wavelengths and intensities to detect the breast tissues at different depths.

The rotative light-emitting/detecting unit 32 of the present invention can continuously emit light located in the near infrared region with predetermined wavelengths and intensities. The wavelengths of the light are located in the region from 320 nm to 500 nm and from 650 nm to 1200 nm, and the intensities thereof are in the range of 5 mW/cm² to 25 mW/cm². Herein, when the motor (not shown in the figure) starts the light-emitting/detecting unit 32 rotating, each node module 33 can receive near infrared red light with several wavelengths. Herein, the left and the right breasts are respectively irradiated with near infrared red light having wavelengths of 680 nm, 760 nm, 805 nm, 850 nm, 910 nm, 970 nm, and 1010 nm. Then, the node module 33 can collect the first signals generated from the breast tissue, and the first signals are transmitted to the photo detector 332 through the photo signal-transmitting unit 332. In the present embodiment, the device can receive the optical characteristics from the position of each node module, to obtain entire photo signals.

In order to improve the detection effect, the material of the cover portion 311 is bamboo charcoal fibers, which has the properties of absorbing the secondary reflection. Hence, the interference from incidental light penetrating through the skin and generating a secondary reflection can be reduced. In addition, the breast health care device of the present invention is designed as female underwear, and the outmost surface of the main body may be a waterproof surface. Hence, the device can be cleaned several times.

### Embodiment 6

The structure of the breast health care device of the present embodiment is almost the same as that disclosed in Embodiment 5, except that the structure of the light-emitting/detecting unit of the present embodiment is different from that of Embodiment 5.

FIG. 15 is a perspective view showing the structure of a rotative light-emitting/detecting unit of Embodiment 6 of the present invention. As shown in FIG. 15, the rotative light-emitting/detecting unit of the present embodiment comprises fourteen light-emitting units 321 such as LEDs, and plural light-transmitting units 331 such as optical fibers and photo signal-transmitting units 332. Herein, seven light-emitting units 321 and heat dispensing modules (not shown in the figure) are arranged side by side, and the light is transmitted to each node module 33 through optical fibers 323. The disposition of the other seven light-emitting units 321 and the heat dispensing modules (not shown in the figure) are arranged in the same way.

When the light-transmitting units 331 of the light-emitting/detecting unit 32 transmit the light to the node modules 33, the first signals (i.e. the light intensities) generated from the breast tissues may transmit back to the light-emitting/detecting unit 32 through the photo signal-transmitting units 332, and the detection can be accomplished by the photo detector 322.

### Embodiment 7

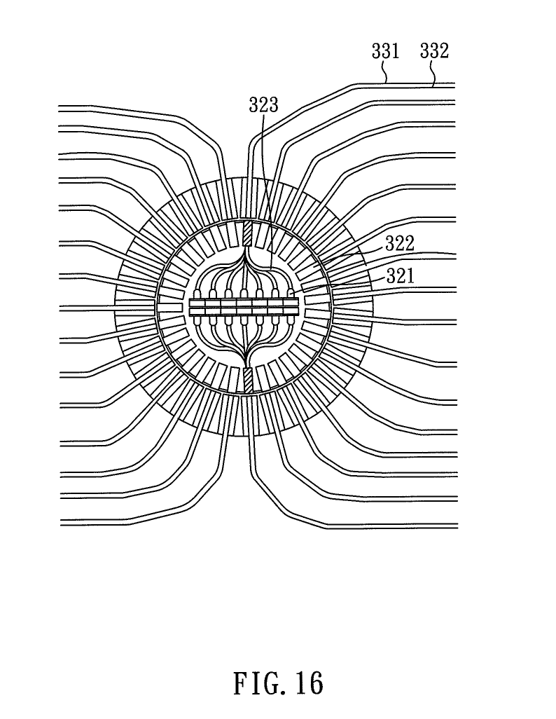

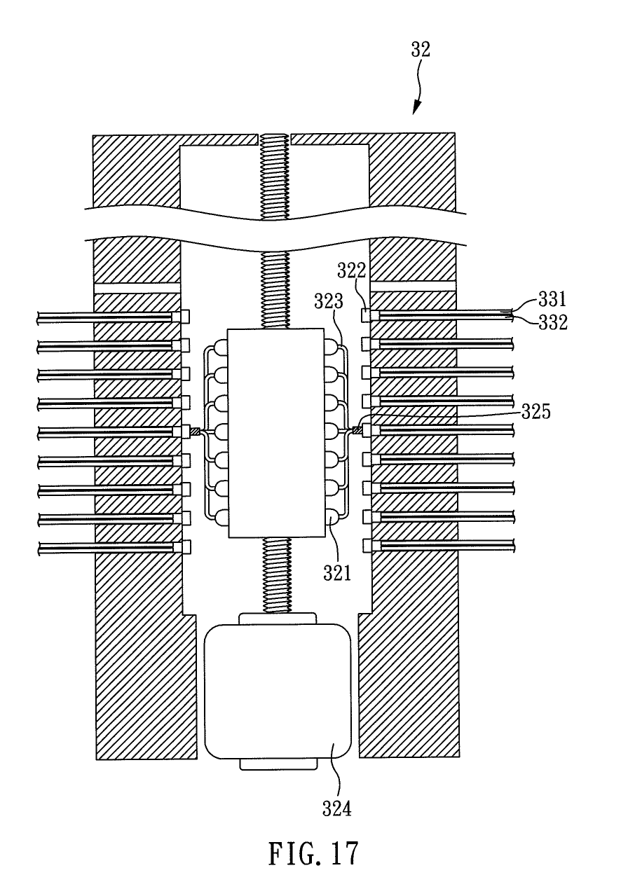

The present embodiment is almost the same as Embodiment 5, except that the structure of the light-emitting/detecting unit is different. In the present embodiment, the light-emitting/detecting unit is a linear reciprocating light-emitting/detecting unit 32. As shown in FIG. 16, the linear reciprocating light-emitting/detecting unit 32 comprises a motor 324, fourteen light-emitting units 321 such as LEDs, a light-emitting focusing lens set 325, plural photo detectors 322 and a photo-detecting focusing lens set (not shown in the figure). Herein, the light-emitting units 321 are combined with a heat dispersing set (not shown in the figure), and seven light-emitting units 321 and the heat dispensing set (not shown in the figure) are arranged side by side. The light is transmitted to the light-emitting units 331 such as optical fibers through the seven optical fibers 323 and the light-emitting focusing lens set 325, and then the light is transmitted to each node module. Finally, the first signal generated from the breast tissues may be transmitted to the photo-detecting focusing lens set (not shown in the figure) through the photo signal-transmitting units 332 of each node module, and the photo detector 322 may detect the first signal.

According to the linear reciprocating light-emitting/detecting unit 32 of the present embodiment, light with different wavelengths and intensities can be transmitted to each light-transmitting unit 331 based on the setting of a time sequence. For example, the light-emitting focusing lens set 325 may move to the next light-transmitting unit 331 at a predetermined time, to transmit light to the node module.

When the linear reciprocating light-emitting/detecting unit 32 is driven by a motor 324, the motor 324 may drive the light-emitting units 321 and the light-emitting focusing lens set 325 moving linearly and reciprocatingly. Hence, the light can be transmitted to each light-transmitting unit 331. Finally, each node module can transmit the first signal generated from the breast tissues back to the photo detector 322 to complete the detection.

Although the present invention has been explained in relation to its preferred embodiment, it is to be understood that many other possible modifications and variations can be made without departing from the spirit and scope of the invention as hereinafter claimed.

What is claimed is:

1. A breast health care device, comprising:
a main body, which comprises at least one cover portion; and
at least one node module disposed on the cover portion, wherein the node module comprises: a light-emitting unit, a photo detector, or a combination thereof, the light-emitting unit provides a light with predetermined wavelength to breast tissues, and the photo detector detects a first signal generated from the breast tissues.

2. The breast health care device as claimed in claim 1, further comprising: at least one processor, which processes the first signal detected by the photo detector to obtain a second signal.

3. The breast health care device as claimed in claim 1, wherein the light-emitting unit is a light emitting diode, a laser light-emitting unit, or a combination thereof.

4. The breast health care device as claimed in claim 2, wherein the processor comprises: an optical simulation unit, which calculates the first signal to obtain the second signal.

5. The breast health care device as claimed in claim 4, wherein the optical simulation unit is a Monte Carlo unit.

6. The breast health care device as claimed in claim 2, further comprising: an information storage module, a signal-transmitting module, or a combination thereof, wherein the information storage module stores the second signal, and the signal-transmitting module transmits the second signal.

7. The breast health care device as claimed in claim 1, further comprising: a suction unit, an attaching unit, or a combination thereof, wherein the suction unit connects to the node module through a pipe to exhaust gas inside the node module, and the attaching unit is disposed on the node module.

8. The breast health care device as claimed in claim 1, wherein the cover portion has a bra cup structure.

9. The breast health care device as claimed in claim 1, further comprising: a waterproof surface disposed on an outmost surface of the main body.

10. A breast health care device, comprising:
a main body, which comprises at least one cover portion;
at least one light-emitting unit disposed on the main body, wherein the light-emitting unit provides a light with a predetermined wavelength to breast tissues; and
at least one node module disposed on the cover portion, wherein the node module comprises: a photo detector, a light-emitting element, or a combination thereof, wherein the light-emitting element transmits light of a predetermined wavelength to the breast tissues, and the photo detector detects a first signal generated from the breast tissues.

11. The breast health care device as claimed in claim 10, wherein the light-transmitting unit is an optical fiber.

12. The breast health care device as claimed in claim 10, further comprising: at least one processor, which processes the first signal detected by the photo detector to obtain a second signal.

13. The breast health care device as claimed in claim 10, wherein the light-emitting unit is a light emitting diode, a laser light-emitting unit, or a combination thereof.

14. The breast health care device as claimed in claim 12, wherein the processor comprises: an optical simulation unit, which calculates the first signal to obtain the second signal.

15. The breast health care device as claimed in claim 14, wherein the optical simulation unit is a Monte Carlo unit.

16. The breast health care device as claimed in claim 12, further comprising: an information storage module, a signal-transmitting module, or a combination thereof, wherein the information storage module stores the second signal, and the signal-transmitting module transmits the second signal.

17. The breast health care device as claimed in claim 10, further comprising: a suction unit, an attaching unit, or a combination thereof, wherein the suction unit connects to the node module through a pipe to exhaust gas inside the node module, and the attaching unit is disposed on the node module.

18. The breast health care device as claimed in claim 10, wherein the cover portion has a bra cup structure.

19. The breast health care device as claimed in claim 10, further comprising: a waterproof surface disposed on an outmost surface of the main body.

20. A breast health care device, comprising:
a main body, which comprises at least one cover portion;
a light-emitting/detecting unit disposed on the main body, wherein the light-emitting/detecting unit provides light with a predetermined wavelength to breast tissues and detects a first signal generated from the breast tissues; and
at least one node module disposed on the cover portion, wherein the node module comprises: a light-transmitting unit, a photo signal-transmitting unit, or a combination thereof, wherein the light-transmitting unit transmits the light with a predetermined wavelength to the breast tissues, and the photo signal-transmitting unit transmits the first signal to the light-emitting/detecting unit.

21. The breast health care device as claimed in claim 20, wherein the light-emitting/detecting unit is a rotative light-emitting/detecting unit, or a linear reciprocating light-emitting/detecting unit.

22. The breast health care device as claimed in claim 20, wherein at least one of the light-transmitting unit and the photo signal-transmitting unit is an optical fiber.

23. The breast health care device as claimed in claim 20, wherein the processor comprises: an optical simulation unit, which calculates the first signal detected by the light-emitting/detecting unit to obtain the second signal.

24. The breast health care device as claimed in claim 23, wherein the processor comprises: an optical simulation unit, which calculates the first signal to obtain the second signal.

25. The breast health care device as claimed in claim 25, wherein the optical simulation unit is a Monte Carlo unit.

26. The breast health care device as claimed in claim 23, further comprising: an information storage module, a signal-transmitting module, or a combination thereof, wherein the information storage module stores the second signal, and the signal-transmitting module transmits the second signal.

27. The breast health care device as claimed in claim 20, further comprising: a suction unit, an attaching unit, or a combination thereof, wherein the suction unit connects to the node module through a pipe to exhaust gas inside the node module, and the attaching unit is disposed on the node module.

28. The breast health care device as claimed in claim 20, wherein the cover portion has a bra cup structure.

29. The breast health care device as claimed in claim 20, further comprising: a waterproof surface disposed on an outmost surface of the main body.
